(19) United States
(12) Patent Application Publication
(10) Pub. No.: US 2003/0125575 A1
(43) Pub. Date: Jul. 3, 2003

(54) Process for the preparation of(R)-2-bromo-3-phenyl -propionic acid

(76) Inventors: Franciscus Alphons Marie Lommen, Horst (NL); Helmut Koller, Regensburg (DE); Herbert Scherubl, Regensburg (DE)

Correspondence Address: Kate H Murashige, Morrison & Foerster, Suite 500, 3811 Valley Centre Drive, San Diego, CA, 92130-2332, US

(21) Appl. No.: 10/203,694

(22) PCT Filed: Feb. 2, 2001
(86) PCT No.: PCT/NL01/00079

(30) Foreign Application Priority Data
Feb. 11, 2000 (NL) 1014353

(51) Int. Cl. C07C 327/32

(52) U.S. Cl. 558/251; 562/426

(57) ABSTRACT

Process for the preparation of (R)-2-bromo-3-phenylpropionic acid starting from D-phenylalanine, sodium nitrite and HBr in an aqueous solution, the reaction being carried out in the presence of a bromide salt, at a temperature between −10 and 30° C.

The total amount of HBr plus bromide salt lies between 3 and 10 equivalents, calculated relative to the amount of D-phenylalanine, preferably between 4 and 8 equivalents, the amount of bromide salt ranging from 0.5 to 7 equivalents, calculated relative to the amount of D-phenylalanine. The bromide salt is preferably formed in situ from HBr and a base, for instance KOH or NaOH. Preferably, the (R)-2-bromo-3-phenylpropionic acid obtained is subsequently converted into (S)-2-acetylthio-3-phenylpropionic acid using thioacetic acid and an organic base, for instance triethylamine. The (S)-2-acetylthio-3-phenylpropionic acid obtained can be converted into a pharmaceutical, in particular an ACE inhibitor, for instance Omapatrilat.

The invention relates to a process for the preparation of (R)-2-bromo-3-phenylpropionic acid starting from D-phenylalanine, sodium nitrite and a bromine compound in an aqueous solution.

Such a process is known from WO-A-99/42431.

A drawback of the known process is that the reaction must be carried out at a low temperature.

The invention aims to eliminate the above-mentioned drawback.

This is achieved according to the invention by carrying out the reaction in the presence of a bromide salt. In the process according to the invention, moreover, less byproduct and a higher yield can be obtained.

(R)-2-bromo-3-phenylpropionic acid is a suitable intermediate in the preparation of pharmaceuticals, for instance in the preparation of ACE inhibitors, for instance Omapatrilat (known under the commercial name Vanlev), or similar pharmaceuticals.

The process according to the invention is carried out in the presence of HBr and of a bromide salt. Examples of suitable bromide salts are alkali metal or alkaline earth metal salts of HBr, for instance NaBr, KBr or CaBr₂. As a rule a more than equivalent amount of Br⁻ (HBr and bromide salt) is used, preferably 3-10 equivalents, more in particular 4-8 equivalents of Br⁻ calculated relative to the total amount of D-phenylalanine. In principle it is possible to use larger amounts of Br⁻, but this does not yield any significant advantage. The amount of bromide salt depends on the desired excess of Br⁻ and preferably lies between 0.5 and 7 equivalents, in particular between 1.5 and 3 equivalents, calculated relative to the total amount of D-phenylalanine.

In a particularly suitable embodiment at least a part of the bromide salt is formed in situ from HBr and a base. Examples of suitable bases that can be used for this purpose are alkali metal hydroxides, carbonates or bicarbonates. Preferably, KOH or NaOH is used as base.

The amount of base to be used depends on the desired excess of Br and the desired amount of bromide salt, and preferably lies between 0.5 and 7, in particular between 1.5 and 3 equivalents, calculated relative to the total amount of D-phenylalanine.

Applicant has found that the process according to the invention can be carried out at higher temperatures than the temperatures described for the prior art processes. At higher temperatures less energy is required. Moreover the reaction can be performed at higher concentration. The conversion of D-phenylalanine into (R)-2-bromo-3-phenylpropionic acid is carried out at a temperature between −10 and 30° C., for instance between −10 and 20° C. preferably between −5 and 20° C., for instance between −5 and 10° C.

The amount of sodium nitrite to be used preferably lies between 0.8 and 2 equivalents, in particular between 1 and 1.6 equivalents of sodium nitrite, calculated relative to the total amount of D-phenylalanine.

The process according to the invention is preferably carried out in the presence of an organic solvent, for instance a hydrocarbon, preferably a (halogenated) aromatic hydrocarbon. Preferably, xylene or toluene is used as organic solvent.

The (R)-2-bromo-3-phenylpropionic acid that is obtained can subsequently, optionally without intermediate isolation, be converted in a known way into (S)-2-acetylthio-3-phenylpropionic acid using thioacetic acid in the presence of an alkali metal carbonate or bicarbonate, for instance sodium carbonate, sodium bicarbonate, potassium carbonate or potassium bicarbonate, or with an alkali metal salt of thioacetic acid. Preferably, however, this reaction is carried out with an organic base instead of an alkali metal (bi)carbonate. Suitable examples of such a base are alkylamines, in particular trialkylamines; heterocyclic amines in particular pyridines; and (alkyl)anilines. Preferably, triethylamine is used. The reason for this is that it has, surprisingly, been found that in this way the amount of by-product obtained is significantly lower and thus the efficiency higher.

In the preparation of (S)-2-acetylthio-3-phenylpropionic acid from (R)-2-bromo-3-phenylpropionic acid the organic base is preferably dosed to a mixture of (R)-2-bromo-3-phenylpropionic acid and thioacetic acid. In principle it is also possible to use a differ rent dosing sequence.

The temperature at which this reaction is carried out preferably lies between −10 and +30° C., in particular between −5 and +10° C.

The amount of thioacetic acid to be added preferably lies between 0.8 and 2 equivalents, in particular between 0.9 and 1,6 equivalents, calculated relative to the total amount of D-phenylalanine; or between 1 and 2 equivalents, in particular between 1.1 and 1.7 equivalents, calculated relative to the total amount of (R)-2-bromo-3-phenylpropionic acid.

The amount of organic base to be added preferably lies between 0.8 and 2 equivalents, in particular between 1 and 1.8 equivalents, calculated relative to the total amount of D-phenylalanine; or between 1 and 2 equivalents, in particular between 1.2 and 1.8 equivalents, calculated relative to the total amount of (R)-2-bromo-3-phenylpropionic acid.

After the reaction the organic base and the excess thioacetic acid can be removed, for instance by extraction at a pH between 0 and 4.

The invention will now be elucidated on the basis of the examples, without however being limited by this.

### EXAMPLE I

### Preparation of R-2-bromo-3-phenylpropionic acid

46.0 ml of water was introduced into a 1-litre double-walled glass reactor connected to a cooling medium.

275.5 g of 48% HBr was added. The jacket cooling and stirring were started. Subsequently, 67.7 g of 45% KOH was slowly added.

The reaction mixture was cooled to 30-40° C.

45.0 g of D-phenylalanine was added to the reaction mixture. Next, 213 ml of toluene was added to the reaction mixture. The reaction mixture was cooled to 3° C.

In 6 hours 95.9 g of NaNO₂, a 30% solution in water, was added to the reaction mixture. The temperature was kept at 5° C. After the reaction stirring was continued for 3 hours at 3° C.

The reaction mixture was heated to 20° C. Stirring was stopped and the aqueous phase was separated off.

Then the toluene phase was extracted twice with 95 ml of water.

The reaction mixture was heated to 70° C. and a 100 mbar vacuum was created using a vacuum pump. The water was removed by distillation using a Dean Stark apparatus until the toluene phase was free of water. Yield: 84.0% R-2-bromo-3-phenylpropionic acid in the toluene solution, relative to D-phenylalanine.

### Preparation of S-acetylthiophenylpropionic acid

The toluene solution of R-2-bromo-3-phenyl-propionic acid prepared from 45.0 g of D-phenylalanine was cooled to 0° C. Next, 27.0 g of thioacetic acid was added.

In 6 hours 38.5 g of triethylamine was dosed to the reaction mixture at a temperature of 0° C.

Then the reaction mixture was heated to 10° C. Stirring was continued for another 4 hours until the conversion as determined by HPLC was complete.

95 ml of water was added to the reaction mixture and the reaction mixture was heated to 20° C.

32% HCl was used to adjust the pH of the reaction mixture to 3.4. Stirring was stopped and the water was separated off.

Then the reaction mixture was washed with 95 ml of sodium thiosulphate solution (5%).

The pH of the reaction mixture was adjusted to 0.75 using 32% HCl. Then the aqueous phase was separated off and the toluene phase was again extracted with 95 ml of water.

With the aid of a Dean Stark apparatus the water was removed by azeotropic distillation at 60° C. and a pressure of 100 mbar until the toluene phase was water-free.

The toluene phase was concentrated to 150 ml by evaporation and filtered at a temperature of about 40° C. At 40° C. 360 ml of special boiling point gasoline 80-110 was-added, followed by cooling to 0° C.

Yield: 410 g≅67.1% relative to D-phenylalanine.

### EXAMPLE II

### Preparation of R-2-bromo-3-phenylpropionic acid

The reaction is performed in a 1-litre double-walled glass reactor connected to a cooling medium provided with a pitch blade turbine stirrer.

72.0 g of D-phenylalanine was introduced into the reactor. 442 g of 48% HBr was added. The jacket cooling and stirring were started. Subsequently, 113 g of 45% KOH was slowly added.

The reaction mixture was cooled to 20° C.

Next, 330 ml of toluene was added to the reaction mixture.

In 5 hours 90.56 ml of NaNO₂, a 34.7 wt % solution in water (39.09 g NaNO₂), was added to the reaction mixture. The reaction mixture was kept at an internal temperature of 20° C. After the reaction stirring was continued for 1 hour at 20° C.

Stirring was stopped and the aqueous phase was separated off.

Then the toluene phase was extracted twice with 160 ml of water.

The reaction mixture was heated to 70° C. and a 100 mbar vacuum was created using a vacuum pump. The water was removed by distillation using a Dean Stark apparatus until the toluene phase was free of water. The toluene phase was concentrated by evaporation at 50° C. and 50 mbar whereby 94.2 g crystallized oil was obtained. Content of R-2-bromo-3-phenylpropionic acid herin was 89.1 wt % corresponding to 84.1 mol % R-2-bromo-3-phenylpropionic acid relative to D-phenylalanine.

1. Process for the preparation of (R)-2-bromo-3-phenylpropionic acid starting from D-phenylalanine, sodium nitrite and HBr in an aqueous solution, characterized in that the reaction is carried out in the presence of a bromide salt, at a temperature between −10 and 30° C. and in the presence of an organic solvent.

2. Process according to claim 1 in which the total amount of HBr plus bromide salt lies between 3 and 10 equivalents, calculated relative to the amount of D-phenylalanine.

3. Process according to claim 2 in which the amount of HBr plus bromide salt lies between 4 and 8 equivalents, calculated relative to D-phenylalanine.

4. Process according to any one of claims 1-3 in which the amount of bromide salt lies between 0.5 and 7 equivalents, calculated relative to the amount of D-phenylalanine.

5. Process according to any one of claims 1-4 in which at least a part of the bromide salt is formed in situ from HBr and a base.

6. Process according to claim 5 in which an alkali metal hydroxide, carbonate or bicarbonate is used as base.

7. Process according to claim 6 in which KOH or NaOH is used as base.

8. Process according to any one of claims 5-7 in which the total amount of base used lies between 0.5 and 7 equivalents relative to the total amount of D-phenylalanine.

9. Process according to any one of claims 1-8 in which the temperature lies between 5° C. and +20° C.

10. Process according to any one of claims 1-9 in which the amount of sodium nitrite lies between 1 and 1.4 equivalents of sodium nitrite, calculated relative to the amount of D-phenylalanine.

11. Process according any of claims 1-10 in which toluene or xylene is used as organic solvent.

12. Process according to any one of claims 1-11 in which the (R)-2-bromo-3-phenylpropionic acid obtained is subsequently converted into (S)-2-acetylthio-3-phenylpropionic acid using thioacetic acid and an organic base.

13. Process according to claim 12 in which an alkylamine, a heterocyclic amine or an (alkyl)aniline is used as organic base.

14. Process according to claim 13 in which triethylamine is used as organic base.

15. Process according to any one of claims 12-14 in which the base is dosed to a mixture of (R)-2-bromo-3-phenylpropionic acid and thioacetic acid at a temperature between −10° C. and +30° C.

16. Process according to any one of claims 12-15 in which the (S)-2-acetylthio-3-phenylpropionic acid obtained is converted into a pharmaceutical, in particular an ACE inhibitor, for instance Omapatrilat.
